(19) United States
(12) Plant Patent Application Publication
(10) Pub. No.: US 2019/0133013 P1
(43) Pub. Date: May 2, 2019

(54) Spiraea plant names 'Minspil04'

(50) ;

(71) Applicant: Patrick Pineau, Saint Mathurin Sur Loire (FR)

(72) Inventor: Patrick Pineau, Saint Mathurin Sur Loire (FR)

(73) Assignee: HORTIVAL DIFFUSION SAS, Beaufort En Anjou (FR)

(21) Appl. No.: 15/998,283

(22) Filed: Jul. 30, 2018

(30) Foreign Application Priority Data
Oct. 27, 2017 (EM) 20172753

(51) Int. Cl. A01H 6/00

(52) U.S. Cl. PLT/226

(57) ABSTRACT

A new cultivar of Spiraea named ‘Minspil04’ that is characterized by compact plant habit, its unique foliage color changes throughout the seasons with leaves that are yellow with new growth flushed with orange in spring and changes to pink-red in fall, and its foliage that has no green reversion.

### CROSS REFERENCE TO A RELATED APPLICATION

This application is related to a European plant breeders' rights application filed on Oct. 27, 2017, application No. 2017/2753 for which priority is claimed. There have been no offers for sale anywhere in the world prior to the effective filing date of this Application and no accessibility to one of ordinary skill in the art could have been derived from the printed plant breeder's rights documents.

### STATEMENT REGARDING PRIOR DISCLOSURES BY THE INVENTOR

The Applicant asserts that no publications or advertisements relating to sales, offers for sale, or public distribution occurred more than one year prior to the effective filing date of this application. Any information about the claimed plant would have been obtained from a direct or indirect disclosure from the Inventor. The Applicant claims a prior art exemption under 35 U.S.C. 102(b)(1) for disclosure and/or sales prior to the filing date but less than one year prior to the effective filing date. Disclosure include but may not be limited to posting on www.sapho.fr and www.pepinieres-minier.fr.

Botanical classification: Spiraea japonica.

Variety denomination: ‘Minspil04’.

### BACKGROUND OF THE INVENTION

The present invention relates to a new and distinct cultivar of Spiraea japonica referred to hereafter by its cultivar name, ‘Minspil04’. ‘Minspil04’ is a new variety of Spiraea grown for landscape use.

The New cultivar was derived from an ongoing breeding program conducted by the Inventor in in La Menitre, France with the goal of developing a new cultivar of Spiraea with a compact plant habit, uniquely colored foliage, and healthy foliage. The new cultivar derived from open pollination of an unnamed unpatented seedling from the Inventor's breeding program in 2010. The Inventor selected ‘Minspil04’ as a single unique plant amongst the seedlings that resulted from the above cross in July of 2012.

Asexual propagation of the new cultivar was first accomplished by the Inventor by softwood stem cuttings in July of 2012 in La Menitre, France. Asexual propagation of the new cultivar by softwood stem cuttings has determined that the characteristics of the new cultivar are stable and are reproduced true to type in successive generations.

### SUMMARY OF THE INVENTION

The following traits have been repeatedly observed and represent the characteristics of the new cultivar, which in combination distinguish ‘Minspil04’ as a new and unique cultivar of Spiraea.
1. ‘Minspil04’ exhibits a compact plant habit.
2. ‘Minspil04’ exhibits unique foliage color changes throughout the seasons with leaves that are yellow with new growth flushed with orange in spring and changes to pink-red in fall.
3. ‘Minspil04’ exhibits foliage that has no green reversion.

The female parent of ‘Minspil04’ differs from ‘Minspil04’ in having leaves that are red in color when emerging, green in color in the spring and summer, and green-purple in color in fall and in having a large, bushy plant habit. ‘Minspil04’ can also be most closely compared to the Spiraea cultivars ‘Goldflame’ (not patented) and ‘Walbuma’ (U.S. Plant Pat. No, 9,363). ‘Goldflame’ differs from ‘Minspil04’ in having green leaf reversion, a larger less compact plant habit, leaves that are red in when expanding and in fall. ‘Walbuma’ differs from ‘Minspil04’ in having a less vigorous growth habit and in having emerging foliage that is more red and less orange in color.

### BRIEF DESCRIPTION OF THE DRAWINGS

The accompanying colored photographs illustrate the overall appearance and distinct characteristics of the new Spiraea. The photographs were taken of a plant three years in age as grown outdoors in a 4.5-liter container in La Menitre, France.

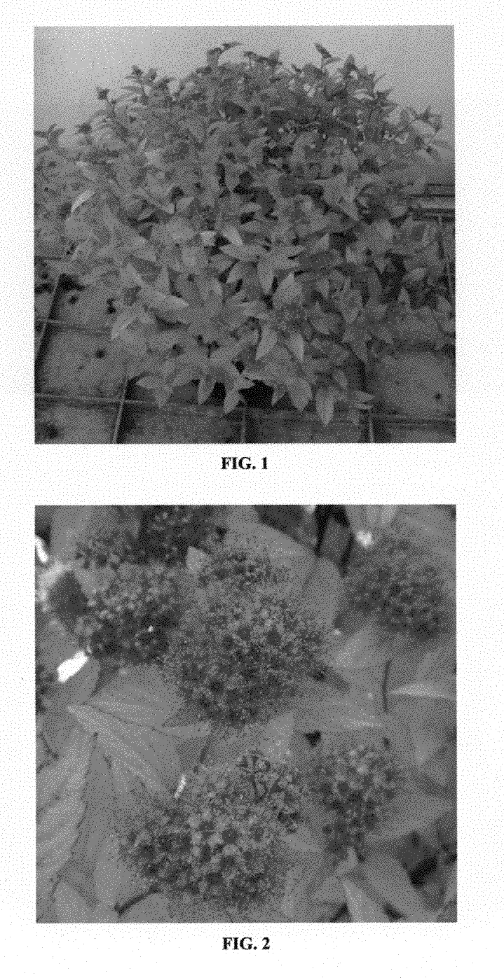

The photograph in FIG. 1 provides a view of the overall plant habit of ‘Minspil04’ in bloom.

The photograph in FIG. 2 provides a close-up view of inflorescences of ‘Minspil04’.

The photograph in FIG. 3 provides a view of the fall color of ‘Minspil04’.

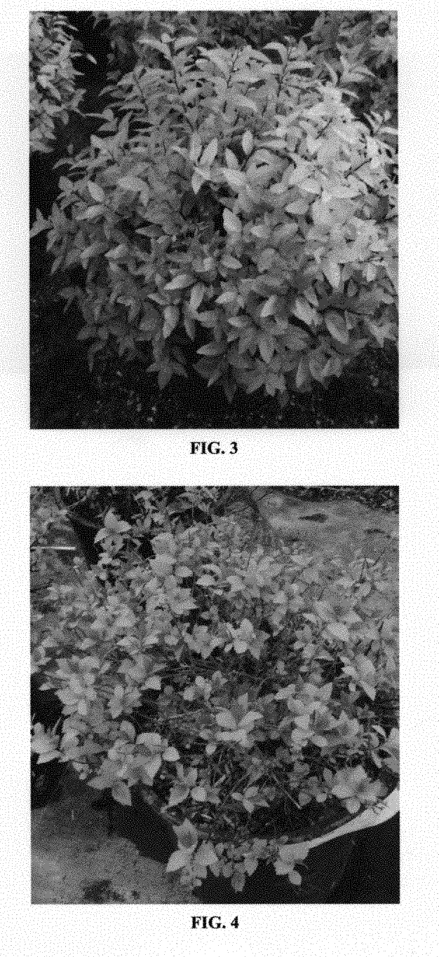

The photograph in FIG. 4 provides a view of the spring foliage of ‘Minspi104’.

The colors in the photographs are as close as possible with the digital photography techniques available, the color values cited in the detailed botanical description accurately describe the colors of the new Spiraea.

### DETAILED BOTANICAL DESCRIPTION

The following is a detailed description of three-year-old plants of the new cultivar as grown outdoors in 2-gallon containers in St. Paul, Minn. The phenotype of the new cultivar may vary with variations in environmental, climatic, and cultural conditions, as it has not been tested under all possible environmental conditions. The color determination is in accordance with the 2015 Colour Chart of The Royal Horticultural Society, London, England, except where general color terms of ordinary dictionary significance are used.
General description:
   Blooming period.—Blooms July, August and September in France.
   Plant type.—Deciduous shrub.
   Plant habit.—Compact, mounded.
   Height and spread.—An average of 53 cm in height and 60 cm width as grown in a 2-gallon container, reaches 0.5 m in height and spread as a mature plant in the landscape.
   Hardiness.—At least in U.S.D.A. Zones 3 to 8.
   Diseases and pests.—No susceptibility or resistance to diseases or pests has been observed.
   Root description.—Fibrous and fine, 200A in color.
   Propagation.—Softwood stem cuttings.
   Growth rate.—Moderate.
   Root initiation.—Average of 7 to 10 days.
   Root development.—Average of 120 days to fully develop in a 2-gallon container.
Branch description:
   Branch shape.—Rounded.
   Branch color.—New growth; 143C in spring and becoming 147A suffused with 183A to 185A in summer, mature wood; 200B to 199D, trunk; 165A.
   Branch size.—Main branches; an average of 32 cm in length and 1 cm in width, lateral branches; an average of 36 cm in length and 5 mm in width, trunk; 2 cm in length, 2 cm in width.
   Branch strength.—Strong.
   Branch surface.—New growth; slightly glossy and smooth, mature; ribbed surface, slightly glossy to dull, covered with thin woolly pubescence that match surface color, mature wood; bark-like, rugose, trunk; smooth, slightly glossy.
   Internode length.—An average of 1 cm.
   Branching.—An average of 12 main stems, an average of 16 lateral branches per main stem, stems held upright to outward in multiple angles.
Foliage description:
   Leaf shape.—Elliptic.
   Leaf division.—Simple.
   Leaf base.—Acute.
   Leaf apex.—Acute.
   Leaf fragrance.—None.
   Leaf venation.—Pinnate, upper and surfaces change to match leaf surface color throughout the seasons.
   Leaf margins.—Serrated.
   Leaf arrangement.—Alternate.
   Leaf attachment.—Petiolate.
   Leaf surface.—Both surfaces glabrous and dull.
   Leaf size.—Average of 5 cm in length and 2 cm in width.
   Leaf quantity.—Average of 38 per lateral branch.
   Leaf color.—Spring; young upper and lower surface 145A, as leaves expand developing to N172A, mature upper and lower surface 180A and 144A, summer; young upper and lower surface 145A, as leaves expand developing to a blend of N167A and 34A, mature upper and lower surface 145A to 143A, some slightly flushed with 167A, fall lower surface; upper portion of plant foliage ranging between 48A, 158A, 45A, 47A and 173A, lower portion of plant foliage 142A to 140A.
   Petioles.—Up to 5 mm in length and 2 mm in width, spring color; 144A suffused with 172A towards the top, summer color; 175A, fall color; 187A, glabrous and satiny surface.
   Stipules.—Glandular-like, an average of 2 mm in length and 1 mm in diameter, dull, flaky surface, changes in color throughout the season to match leaf color surfaces.
Inflorescence description:
   Inflorescence type.—Dense umbellate corymbs, present at terminus of lateral branches.
   Inflorescence size.—Average of 7 cm in diameter and 5 cm in depth.
   Peduncles.—Average of 1.5 cm in length and 1 mm in width, 138A in color, surface is matte and sparsely covered with pubescence, moderately strong.
   Pedicels.—An average of 2 mm in length and 0.5 mm in width, 138A in color, surface is matte and sparsely covered with pubescence, moderately strong.
   Flower buds.—Globose in shape, 1 mm in depth and diameter, 182A in color.
   Flower fragrance.—None.
   Persistence of flowers.—Calyx persistent.
   Flower quantity.—Average of 55 per inflorescence.
   Lastingness of inflorescence.—An average of 10 days with individual flowers lasting an average of 2 days.
   Flower type.—Rotate with numerous extended stamens.
   Flower aspect.—Inflorescence held upright on stem terminus.
   Flower size.—Average of 5 mm in diameter and 3 mm in depth.
   Petals.—5, average of 2 mm in length and width, orbicular in shape, apex is wavy and slightly notched, rounded base, both surfaces glabrous, close to 68A in color when opening, when fully open N75A and N82A to N82B, not persistent.
   Calyx.—Cone-shaped with flared tips, average of 3 mm in width and 2 mm in depth.
   Sepals.—5, tips are an average of 0.5 mm in length and 1 mm in width, fused base, ovate in shape, entire margins, acute apex, 142C, margins 145B in color, surface glabrous on both surfaces.
Reproductive organs:
   Pistils.—5, style; 2 mm in length and a blend of 185A at the tips and 76B at the base in color, stigmas; 0.5 mm in diameter, 185A in color, ovaries; superior, oblong in shape, 0.5 mm in length and 0.4 mm in width, 142C in color.
   Stamens.—Average of 15, showy filaments; up to 5 mm in length and NN155A in color, anthers; 0.3 mm in diameter, 164A in color, pollen; low in quantity, NN155B in color.
   Fruit and seed.—None observed to date.

It is claimed;

1. A new and distinct cultivar of Spiraea plant named ‘Minspil04’ as herein illustrated and described.
